(19) United States
(12) Patent Application Publication
(10) Pub. No.: US 2017/0290605 A1
(43) Pub. Date: Oct. 12, 2017

(54) INTRACAVITARY MEDICAL DEVICES AND METHODS FOR USING SAME

(71) Applicant: Younes Noaman Bakri, Amman (JO)

(72) Inventor: Younes Noaman Bakri, Amman (JO)

(21) Appl. No.: 15/485,197

(22) Filed: Apr. 11, 2017

(30) Foreign Application Priority Data
Apr. 11, 2016 (JO) 64/2016

(51) Int. Cl. A61B 17/42; A61M 25/10; A61B 5/15; A61M 31/00; A61M 3/02; A61B 10/02; A61M 25/00; A61M 27/00

(52) U.S. Cl. CPC A61B 17/42; A61M 25/0026; A61M 25/10; A61M 27/00; A61M 31/00; A61M 3/0295; A61M 3/0283; A61B 10/0291; A61B 5/150992; A61M 2210/1433; A61M 2210/1475; A61B 2017/4216

(57) ABSTRACT

The invention generally relates to intracavitary medical devices and methods for using the devices. In one aspect, the devices comprise: a tube comprising a longitudinal body having opposed proximal and distal ends, and at least three lumens extending there between; and an expandable balloon region located at the distal end of the tube; wherein at least one lumen comprises at least one of: a drainage lumen which opens at a portion of the distal end of the tube and configured to allow fluid communication from a body cavity into the drainage lumen, an inflation lumen connected to an interior of the balloon region and configured to enable passage of a distending fluid to control expansion and contraction of the balloon region, and an instillation lumen which opens at a portion of the distal end of the tube and configured to allow a fluid to be introduced from the instillation lumen into the body cavity.

### CROSS REFERENCE TO RELATED APPLICATIONS

This claims the benefit of priority to Jordanian Patent Application No. 64/2016, filed Apr. 11, 2016, which is hereby incorporated herein by reference in its entirety.

### FIELD OF INVENTION

This disclosure relates to intracavitary devices and methods for using the devices to treat various disorders and conditions.

### BACKGROUND

Worldwide, obstetric hemorrhage and sepsis/infection are recognized as leading causes of maternal morbidity and mortality. In 2015, about 830 women per day died due to complications of pregnancy and childbirth. Almost all of these deaths occurred in low resource settings, and most could have been prevented. The primary causes of death were hemorrhage, hypertension, infections and other indirect causes, mostly due to interactions between pre-existing medical conditions and pregnancy.

Most cases of severe PPH are preventable if appropriate and timely standard treatment measures are applied. This can be achieved by medical treatments using pharmaceutical uterotonics, which if fail, a second line treatment can be applied, including: uterus tamponade, angiographic embolization, open laparotomy B-Lynch sutures, B-LUVS, uterine artery ligation, hypogastric artery ligation, or hysterectomy as deemed appropriate.

The last two decades has witnessed extensive global collaborative efforts to face the PPH challenge at all possible fronts, including public health, clinical, academic, administrative, socio-economic and other measures. On the clinical front, uterine tamponade has proved to be an effective second line treatment measure, when available and when applied in the appropriate clinical setting. Recently, balloon technology, such as in the “Bakri balloon”, has been used to tamponade the postpartum uterus to control hemorrhage.

Successful outcomes (defined as hemorrhage control without further need for additional more invasive treatment measures such as embolization, open laparotomy, B-lynch, B-LUVS sutures, uterine vessel ligation, hypogastric artery ligation, hysterectomy). Significantly, these failures, with the exception of embolization, can require an open laparotomy surgery. Reported complications associated with treatment failures have included migration/expulsion of balloon, rupture/leakage, uterus perforation and/or infection.

Thus, there remains a need for devices and methods that overcome disadvantages, deficiencies and shortcomings of the currently available tamponade technical designs and devices that effectively treat uterine hemorrhage and sepsis/infection conditions and disorders which are pregnancy-related. This need and other needs are met by the new technology, design and the multiple functions of the present invention.

### SUMMARY

In accordance with the purpose(s) of the invention, as embodied and broadly described herein, the invention, in one aspect, relates to intracavitary devices and methods of using the devices to treat various disorders, such as, uterine and pregnancy-related disorders.

Disclosed are intracavitary medical devices for insertion into a body cavity, the devices comprising: a tube comprising a longitudinal body having opposed proximal and distal ends, and at least three lumens extending there between; and an expandable balloon region located at the distal end of the tube; wherein at least one lumen comprises at least one of: a drainage lumen which opens at a portion of the distal end of the tube and configured to allow fluid communication from a body cavity into the drainage lumen, an inflation lumen connected to an interior of the balloon region and configured to enable passage of a distending fluid to control expansion and contraction of the balloon region, and a one way infusion-instillation lumen which opens at a portion of the distal end of the tube and configured to allow a fluid to be introduced from the infusion-instillation lumen into the body cavity.

Also disclosed are methods for treating a disorder in a subject, the methods comprising: inserting and/or positioning a disclosed medical device within a body cavity; and performing at least one of the following steps: expanding a balloon region of the device within the body cavity, draining a fluid from the body cavity into a drainage lumen of the device, and instilling a fluid or medication through the instillation lumen of the device into the body cavity, thereby treating the subject for the uterine disorder.

Also disclosed are kits comprising the devices.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows a depiction of an intracavitary medical device in accordance with an exemplary embodiment of the present invention.

FIG. 2 shows a depiction of an intracavitary medical device in accordance with an exemplary embodiment of the present invention.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present devices, compounds, compositions, articles, systems, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a lumen,” includes of two or more lumens.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In some aspects of the disclosed methods, the subject has been diagnosed with a need for treatment of one or more uterine associated disorders prior to the administering step. In some aspects of the disclosed method, the subject has been diagnosed with Gestational Trophoblastic Disease prior to the administering step. In some aspects of the disclosed method, the subject been diagnosed with Septic Abortion, Unsafe Abortion Sepsis/Infection. Septic Postpartum Endometritis, Miscarriage Sepsis/Infection Puerperal Sepsis, Postpartum Sepsis. Postabortal Sepsis, Postabortion Hemorrhage, Post-miscarriage Hemorrhage, Molar Pregnancy Hemorrhage, Post Hydatidiform Mole Hemorrhage, Acute Uterine Inversion, Recurrent Uterine Inversion, or the like.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e g, a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a uterine disorder prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “uterine disorders” refers to a condition, disease or disorder characterized by or exhibiting a uterine-related dysfunction. In further various aspects, the dysfunction is uterine bleeding. In further various aspects, a uterine disorder can comprise uterine atony, abortion-miscarriage, placenta previa, recurrent uterine inversion, gestational trophoblastic disease, or a hyperproliferative disorder, such as uterine cancer.

The term “pharmaceutically acceptable” describes a material that is not biologically or otherwise undesirable, i.e., without causing an unacceptable level of undesirable biological effects or interacting in a deleterious manner.

The terms “proximal” and “proximally” are used herein to refer to a position or direction away from, or even external to a patient's body and the terms “distal” and “distally” are used to refer to a position or direction towards the patient and/or to be inserted into a patient's body orifices or cavities.

As used herein, the term “catheter” may be used interchangeably with “tube”, and refers to any tube that can be inserted into a body cavity, duct, or vessel.

Certain materials, compounds, compositions, and components disclosed herein can be obtained commercially or readily synthesized using techniques generally known to those of skill in the art.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. Intracavitary Devices and Methods

In one aspect, the invention relates to intracavitary medical devices for insertion into a body cavity, such as a uterine cavity. More specifically, in one aspect, the present invention relates to intracavitary medical devices for the intrauterine treatment of uterine disorders.

In further aspects, the device comprises: a tube comprising a longitudinal body having opposed proximal and distal ends, and at least three lumens extending therebetween; and an expandable balloon region located at the distal end of the tube. In still further aspects, the at least one lumen comprises at least one of: a drainage lumen which opens at a portion of the distal end of the tube and configured to allow fluid communication from a body cavity into the drainage lumen, an inflation lumen connected to an interior of the balloon region and configured to enable passage of a distending fluid to control expansion and contraction of the balloon region, and an instillation lumen which opens at a portion of the distal end of the tube and configured to allow a fluid to be introduced from the instillation lumen into the body cavity.

In various aspects, the device is configured for insertion into a body cavity. In further aspects, the tube body's distal end can be round and blunt, which can help prevent perforation injury of the uterine wall during tube insertion. In still further aspects, the device can be configured to maintain a shape that conforms with the anatomical shape of the body cavity. In yet further aspects, at least a portion of the tube body can comprise a curved shape, a crescent shape, or an S shape, or a combination thereof.

In further aspects, at least a portion of the tube body can be comprised of a firm or rigid material. In still further aspects, the device components can be comprised of a firm or rigid material. In yet further aspects, a portion of the tube can be comprised of a firm material and other portions can be comprised of a soft or flexible material. In even further aspects, a middle portion of the tube can be comprised of a rigid material. For example, the portion of the tube corresponding to a shape that conforms with the anatomical shape of a birth canal can comprise a rigid material. Previous designs of balloon tamponade devices are generally flexible and have soft main catheter/tube/shaft which do not help in preventing “balloon expulsion” or unintended evacuation of the device. The design, configuration, and composition of the present device can help avoid expulsion by being comprised of a firm material, and having a curved shape (crescent shape), in various aspects, conforming to the birth canal shape. The curved shape of the body can help prevent expulsion of the tube or catheter which happens in approximately 10% of clinical cases leading to failure of tamponade. To this end, expulsion of the balloon leading to failure of tamponade can lead to continuing of the bleeding.

In further aspects, one or more of the device components can be comprised of latex, silicone, rubber latex, silicone coated latex, polyvinyl chloride, or polyurethane, or combinations thereof. In still further aspects, the catheter or tube body can have a length in the range of from about 20 cm to about 100 cm. In even further aspects, the catheter or tube body can have a diameter in the range of from about 1 Fr to about 100 Fr, including exemplary sizes of 24, 28, 32, and 48 Fr. In yet further aspects, the catheter or tube body diameter can be adjusted to accommodate an individual patient specific clinical condition.

In further aspects, each lumen can comprise at least one port located at one end. In still further aspects, at least one lumen can comprise at least one port or opening located at both the proximal end and distal end. In yet further aspects, each lumen can comprise a separate proximal opening (e.g., outside the body cavity). In yet further aspects, the diameter of the port can be from about 0.1 mm to about 10 mm.

In further aspects, a lumen can have a distal opening (e.g., to be located inside the body cavity, and a proximal opening (e.g., accessible outside the body cavity). In further aspects, there can be a separate port for filling the balloon region with a distending fluid, and a separate port for irrigation, lavage and pharmaceutical medications topical treatment by instillation, injection, infusion, or the like. In some aspects, each port can be independent of and separate from the other ports.

In further aspects, the diameter of the at least one lumen can be from about 0.1 mm to about 10 mm. In still further aspects, the diameter of the drainage lumen can be from about 1 mm to about 10 mm. In yet further aspects, the drainage lumen can comprise a drainage port, designed for one-way out exit drain. In even further aspects, the opening of the drainage lumen comprises a Murphy's eye opening. In some aspects, the diameter can be about 10 mm, and can be configured to serve as a passage out for collected fluid and blood in the body cavity.

In further aspects, the lumen can comprise an instillation or infusion port for topical pharmaceutical composition instillation/injection/infusion treatment. In still further aspects, the pharmaceutical composition can comprise an effective amount of at least one compound selected from: an agent known to treat an infection or a pharmaceutically acceptable salt thereof; and an agent known to modulate the immune system or pharmaceutically acceptable salt thereof, an agent known to modulate hemostasis or pharmaceutically acceptable salt thereof, an agent known to treat cancer or pharmaceutically acceptable salt thereof, an agent known to modulate hormones or pharmaceutically acceptable salt thereof, an agent used for topical or local treatment intracavitary or intrauterine, or transcervical or transvaginal, for example, and without limitation, uterotonics, antibiotics, tranexamic acid, and other agents known to treat uterine disorders.

In further aspects, a port can be configured as a one-way port directed towards or away from the body cavity. To this end, one-way direction can help avoiding infection when compared to two-way direction.

In further aspects, the device can comprise an inflation port which directs distending fluid to fill the balloon region. In some aspects, the inflation port and lumen connect directly to inside the balloon region only, and not to the body cavity.

In further aspects, the device can comprise a drainage opening connected to a separate port for drainage, for example, to remove irrigation-lavage fluids in addition to bleeding which exits out of the patient body in one-way direction. In still further aspects, the device can comprise an instillation opening at the distal end, which can be connected to the instillation port by a lumen, and can deliver pharmaceutical composition treatments and/or lavage solutions, directly into the uterine cavity, for example, through a distal instillation opening configured for that purpose.

In further aspects, the balloon region is configured to provide pressure tamponade. This balloon region, when filled, takes the anatomical shape, size and volume of the body cavity, such as the uterine cavity, which is variable in individual patient case. This variation in uterine cavity size and volume, in patient individual case has not been taken into consideration in currently available balloon tamponade devices. This variation in uterine cavity size and volume has been taken into consideration in the disclosed devices. The inventive devices of the present disclosure can comprise variable catheter or tube body sizes to fit the approximate size-diameter of the uterine cavity according to clinical condition of individual patient cases. Moreover, the disclosed device can have variable size and variable volumes of its tamponade balloon region to fit and match the variable size and volume of the individual patient's uterine cavity.

In various aspects, the balloon region can be configured to be inflated with the distending fluid to a maximum volume from about 50 milliliters to about 750 milliliters. In further aspects, the balloon region can have a predetermined size and a predetermined maximum volume. In still further aspects, the catheter or tube body can have a predetermined size and a predetermined diameter. In some aspects, the catheter or tube body has a predetermined size and a predetermined diameter; and the balloon region has a predetermined size and a predetermined maximum volume.

In further aspects, the predetermined size of the tube-catheter body can be from about 12 French gauge to about 48 French gauge. In still further aspects, the predetermined diameter of the catheter or tube body can be from about 28 French to about 32 French. In yet further aspects, the predetermined size of the balloon region can be from about 20 ml to about 750 ml. In even further aspects, the predetermined maximum volume of the balloon region is from about 1 ml to about 50 ml. In still further aspects, the predetermined maximum volume of the balloon region is from about 51 ml to about 150 ml. In yet further aspects, the predetermined maximum volume of the balloon region is from about 151 ml to about 400 ml. In even further aspects, the predetermined maximum volume of the balloon region is from about 401 ml to about 750 ml.

It is understood that the disclosed devices can be employed in the disclosed methods of using.

In various aspects, the disclosed devices can be configured to treat a body cavity disorder, such as, for example, a uterine disorder. In some aspects, the invention relates to methods for treating a body cavity disorder using a disclosed medical device. In other aspects, the invention relates to methods for treating a uterine disorder using a disclosed medical device. In one aspect, the method comprises inserting and/or positioning a disclosed device according to the present invention within a body cavity; and performing at least one of the following steps: expanding the balloon region within the body cavity, draining a fluid from the body cavity into the drainage lumen, and instilling a fluid or medication through the instillation lumen into the body cavity, thereby treating the subject for the body cavity disorder.

In some aspects, the invention relates to a method for the treatment of a subject, the method comprising the steps of: a) diagnosing the subject as having a body cavity disorder; and b) inserting and/or positioning a disclosed device according to the present invention within a body cavity; and performing at least one of the following steps: expanding the balloon region within the body cavity, draining a fluid from the body cavity into the drainage lumen, and instilling a fluid or medication through the instillation lumen into the body cavity; and thereby treating the subject for the body cavity disorder.

In other aspects, the invention relates to a method for the treatment of a subject, the method comprising the steps of: a) diagnosing the subject as having a uterine disorder; and b) inserting and/or positioning a disclosed device according to the present invention within a body cavity; and performing at least one of the following steps: expanding the balloon region within the body cavity, draining a fluid from the body cavity into the drainage lumen, and instilling a fluid or medication through the instillation lumen into the body cavity; and thereby treating the subject for the uterine disorder.

In further aspects, inserting can comprise inserting the distal end of the catheter into the body cavity. The empty balloon region can then be filled with a distending fluid by injecting fluid through the proximal inflation port, which ends at the opening of the balloon. The filled balloon region can be configured to tamponade the inner wall of the cavity to pressure control a bleeding source of blood vessels. In further aspects, the filled balloon region can be configured to direct blood and/or fluid collecting within the body cavity to exit through the distal drainage opening of the drainage lumen, which can be one-way out direction outside a patient body. In some aspects, by creating a pressure tamponade to control bleeding and at the same time, can force collection of bleeding fluid into the drainage opening of the catheter.

In further aspects, the device can treat uterine or other body cavity disorders through topical treatment or instillation of a fluid having one or more pharmaceutical compositions into the body cavity. In still further aspects, topical treatment of the body cavity can be by instilling or infusing a pharmaceutical medication and/or lavage solutions using an instillation port connected to the instillation lumen and the distal instillation opening, directly into the body cavity. Topical treatment is not believed to be described in currently available uterine tamponade balloons, wherein the topical medication is delivered directly into the uterine cavity.

In further aspects, the drainage lumen and/or drainage port can be exclusively configured for the function of one way exiting and draining of fluid collections, and not for irrigation-lavage or any 2-way directional fluid movement. As such, the drainage port can be configured as one-way direction from “inside-out”, while the separate topical medications and lavage/irrigation can be administered through the instillation port configured as one-way “outside-in” direction. This configuration can help reduce risks of introducing infections into the uterine cavity.

In various aspects, the device can be configured to provide at least one of the following functions: pressure-tamponade homeostasis function, drainage, lavage-irrigation, topical treatment, intracavitary instillation, intracavitary infusion, intracavitary drug delivery, topical drug delivery, intrauterine drug delivery, intrauterine drug treatment, and combination thereof. In further aspects, pressure-tamponade homeostasis can comprise controlling bleeding originating from the uterine cavity. In still further aspects, drainage can comprise removing fluid and/or blood collection from the body cavity, such as to prevent infection. In even further aspects, the device can be configured to perform various diagnostic functions. In yet further aspects, the device can be configured to monitor bleeding status, and/or to evaluate success or failure of internal compression treatment (Tamponade Test). In still further aspects, lavage-irrigation can comprise instilling or infusing a fluid into the body cavity, such as, to facilitate clearing of fluid and/or blood collecting in the body cavity, clearing blood clots, and/or help preventing and treating infections inside the uterine cavity.

In further aspects, topical treatment can comprise injection, infusion, and/or instillation of a pharmaceutical compositions and/or medicated solutions into the uterine cavity, for example, as indicated by the individual patient clinical condition.

The devices of the present invention are configured to have multiple sizes and variations options, to accommodate individual patient clinical condition. As described herein, for the catheter body component variation, multiple and variable sizes and diameters, and for the balloon region variation, multiple and variable sizes and volumes.

This feature accommodates matches and fits individual patient clinical status and needs, such as cervix dilation and placental location. Currently available devices erroneously assume that “One Size fits all”. For example, the inventive devices and methods can use patient factors and diagnosis, such as whether the patient had a term pregnancy birth or a miscarriage or abortion.

In further aspects, the method can comprise selecting a predetermined size and/or diameter of catheter body based on the diagnosis. In still further aspects, the method can further comprise selecting a predetermined size and/or volume of balloon region based on the diagnosis. In some aspects, the method comprises selecting a predetermined size and/or diameter of catheter body based on the diagnosis; and selecting a predetermined size and/or volume of balloon region based on the diagnosis.

To treat or control the uterine disorder, the devices are used on a subject in need thereof, such as a vertebrate, e.g., a mammal. The subject can be a human, non-human primate, horse, cow, pig, dog, sheep, goat, or cat. The subject is preferably a mammal, such as a human. In a further aspect, the mammal is a human. In a yet further aspect, the mammal is a horse. In a still further aspect, the mammal is a cow. Prior to using the device, the subject can be diagnosed with a need for treatment of a uterine disorder, such as uterine bleeding.

In various aspects, the devices can be used to administer pharmaceutical compounds or compositions to the subject according to any method. Such methods are well known to those skilled in the art and include, but are not limited to, topical administration, intracavitary administration, intrauterine administration, intravaginal administration, rectal administration, and the like. Administration can be continuous or intermittent. A preparation can be administered therapeutically; that is, administered to treat an existing disease or condition. A preparation can also be administered prophylactically; that is, administered for prevention of a disease or condition.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of topical administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

In a further aspect, the method can comprise the step of identifying a subject in need of treatment of the uterine disorder. In a still further aspect, the subject has been diagnosed with a need for treatment of the uterine disorder prior to the administering step. In an even further aspect, the subject has been diagnosed with a uterine dysfunction prior to the administering step.

In one aspect, diagnosis of a uterine disorder can comprise performing an experiment upon the subject and identifying a level of a biological marker. In a further aspect, diagnosing can comprise determining, in a patient, levels of a marker indicative of a state of the patient, the state being predictive as to whether the patient will manifest reduced symptoms in response to a treatment.

In a further aspect, the biological marker is human chorionic gonadotropin. In a still further aspect, the subject is a biological sample. In a still further aspect, the biological sample is selected from a cell, blood, urine, or tissue.

In one aspect, diagnosis of a uterine disorder comprises a medical history. In a further aspect, the diagnosis comprises comparing the findings of the medical history with the diagnostic standards. In a still further aspect, the diagnosis comprises finding of a uterine dysfunction. In a yet further aspect, the dysfunction is uterine bleeding. In an even further aspect, the dysfunction is uterine sepsis/infection. In further aspects, the uterine disorder can comprise uterine bleeding, postpartum hemorrhage, uterine atony, abortion-miscarriage, placenta previa, recurrent uterine inversion, gestational trophoblastic disease, a hyperproliferative uterine disorder, uterine cancer, hydatidiform, molar pregnancy, chorioamnionitis, endomyometritis, metritis, peritonitis, pelvic inflammatory disease, parametritis, cervicitis, cervical cancer, endometrial cancer, intrauterine infection, pyometra, hematometra, septic abortion, puerperal sepsis, retained blood syndrome (RBS) or a combination thereof.

In a further aspect, the effective amount is a therapeutically effective amount. In a still further aspect, the effective amount is a prophylactically effective amount.

In further aspects, the pharmaceutical composition can comprise an effective amount of at least one compound selected from: an agent known to treat an infection or a pharmaceutically acceptable salt thereof; and an agent known to modulate the immune system or pharmaceutically acceptable salt thereof, an agent known to modulate hemostasis or pharmaceutically acceptable salt thereof, an agent known to treat cancer or pharmaceutically acceptable salt thereof, an agent known to modulate hormones or pharmaceutically acceptable salt thereof, an agent used for topical or local treatment intracavitary or intrauterine, or transcervical or transvaginal.

In a further aspect, the agent known to treat an infection is an antiviral or antibiotic. In a still further aspect, the antiviral can comprise oseltamivir, zanamavir, amantidine, rimantadine, ribavirin, gancyclovir, valgancyclovir, foscavir, Cytogam® (Cytomegalovirus Immune Globulin), pleconaril, rupintrivir, palivizumab, motavizumab, cytarabine, docosanol, denotivir, cidofovir, or acyclovir. In a yet further aspect, the antibiotic can comprise a macrolide (e.g., azithromycin, clarithromycin and erythromycin), a tetracycline (e.g., doxycycline, tigecycline), a fluoroquinolone (e.g., gemifloxacin, levofloxacin, ciprofloxacin and mocifloxacin), a cephalosporin (e.g., ceftriaxone, defotaxime, ceftazidime, cefepime), a penicillin (e.g., amoxicillin, amoxicillin with clavulanate, ampicillin, piperacillin, and ticarcillin) optionally with a β-lactamase inhibitor (e.g., sulbactam, tazobactam and clavulanic acid), such as ampicillin-sulbactam, piperacillin-tazobactam and ticarcillin with clavulanate, an aminoglycoside (e.g., amikacin, arbekacin, gentamicin, kanamycin, neomycin, netilmicin, paromomycin, rhodostreptomycin, streptomycin, tobramycin, and apramycin), a penem or carbapenem (e.g. doripenem, ertapenem, imipenem and meropenem), a monobactam (e.g., aztreonam), an oxazolidinone (e.g., linezolid), vancomycin, glycopeptide antibiotics (e.g. telavancin), or tuberculosis-mycobacterium antibiotics.

In a further aspect, the agent known to modulate the immune system is selected from a corticosteroid, a TNF inhibitor, an immunosuppressant, and a monoclonal antibody. In a still further aspect, the corticosteroid is selected from budesonide, cortisone, dexamethasone, hydrocortisone, methylprednisolone, prednisolone, prednisone, triamcinolone, beclomethasone, budesonide, ciclesonide, flunisolide, fluticasone, mometasone, and triamcinolone. In yet a further aspect, the TNF inhibitor is selected from adalimumab, certolizumab, etanercept, golimumab, and infliximab. In an even further aspect, the immunosuppressant is selected from cyclosporine, azathioprine, basiliximab, aclizumab, muromonab, tacrolimus, glatiramer acetate, mycopehnolate, and sirolimus.

In a further aspect, the agent known to modulate the immune system include compounds that inhibit/decrease cell signaling by inflammatory molecules like cytokines (e.g., IL-1, IL-4, IL-5, IL-6, IL-9, IL-13, IL-18 IL-25, IFN-α, IFN-β, and others), CC chemokines CCL-1-CCL28 (some of which are also known as, for example, MCP-1, CCL2, RANTES), CXC chemokines CXCL1-CXCL17 (some of which are also know as, for example, IL-8, MIP-2), growth factors (e.g., GM-CSF, NGF, SCF, TGF-β, EGF, VEGF and others) and/or their respective receptors. In a still further aspect, the agent known to modulate the immune system is selected from ABN912 (MCP-1/CCL2, Novartis AG), AMG761 (CCR4, Amgen Inc), Enbrel® (TNF, Amgen Inc, Wyeth), huMAb OX40L GENENTECH (TNF superfamily, Genentech Inc, AstraZeneca PLC), R4930 (TNF superfamily, Roche Holding Ltd), SB683699/Firategrast (VLA4, Glaxo SmithKline PLC), CNT0148 (TNFa, Centocor, Inc, Johnson & Johnson, Schering-Plough Corp); Canakinumab (IL-Iβ, Novartis); Israpafant MITSUBISHI (PAF/IL-5, Mitsubishi Tanabe Pharma Corporation); IL-4 and IL-4 receptor antagonists/inhibitors: AMG317 (Amgen Inc), BAY 169996 (Bayer AG), AER-003 (Aerovance), APG-201 (Apogenix); IL-5 and IL-5 receptor antagonists/inhibitors: MEDI563 (AstraZeneca PLC, Medimmune, Inc), Bosatria® (GlaxoSmithKline PLC), Cinquil® (Ception Therapeutic), TMC120B (Mitsubishi Tanabe Pharma Corporation), Bosatria (GlaxoSmithKline PLC), Reslizumab SCHERING (Schering-Plough Corp); MEDI528 (IL-9, AstraZeneca, Medimmune, Inc); IL-13 and IL-13 receptor antagonists/inhibitors: TNX650 GENENTECH (Genentech), CAT-3M (AstraZeneca PLC, Medimmune), AMG-317 (Takeda Pharmaceutical Company Limited), MK6105 (Merck & Co Inc), IMA-026 (Wyeth), IMA-638 Anrukinzumab (Wyeth), MILR1444A/Lebrikizumab (Genentech), QAX576 (Novartis), CNTO-607 (Centocor), MK-6105 (Merck, CSL); Dual IL-4 and IL-13 inhibitors: AIR645/ISIS369645 (ISIS Altair), DOM-0910 (Glaxo SmithKline, Domantis), Pitrakinra/AEROO1/Aerovant™ (Aerovance Inc), AMG-317 (Amgen). In further aspects, immunotherapeutic agents, such as antibodies and immunomodulators, which include, but are not limited to, HERCEPTINS, RITUXANS, OVAREX™, PANOREX@, BEC2, IMC-C225, VITAMIN, CAMPATH@ I/H, Smart MI95, LYMPHOCIDE™, Smart I D10, and ONCOLYM™, rituximab, gemtuzumab, or trastuzumab.

In a further aspect, examples of anti-cancer agents that can be used in the various aspects disclosed herein, including pharmaceutical compositions and dosage forms disclosed herein, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Hormonal therapeutic agents can comprise hormonal agonists, hormonal antagonists (e.g., flutamide, tamoxifen, leuprolide acetate (LUPRON™), LH-RH antagonists), inhibitors of hormone biosynthesis and processing, steroids (e. g., dexamethasone, retinoids, betamethasone, cortisol, cortisone, prednisone, dehydrotestosterone, glucocorticoids, mineralocorticoids, estrogen, testosterone, progestins), antigestagens (e. g., mifepristone, onapristone), antiandrogens (e. g., cyproterone acetate), and the like.

In one aspect, the invention relates to a kit comprising a disclosed device according to the present invention; and instructions for using the device in connection with a uterine disorder. The instructions for using comprise can comprise any step in a disclosed method.

The kits can also comprise compounds co-packaged, co-formulated, and/or co-delivered with other components. For example, a drug manufacturer, a drug reseller, a physician, a compounding shop, or a pharmacist can provide a kit comprising a disclosed device and another component or medication for delivery to a patient.

In a further aspect, the device and the component or medication are co-packaged.

It is understood that the disclosed kits can be prepared from the disclosed devices, compounds, products, and pharmaceutical compositions. It is also understood that the disclosed kits can be employed in connection with the disclosed methods of using.

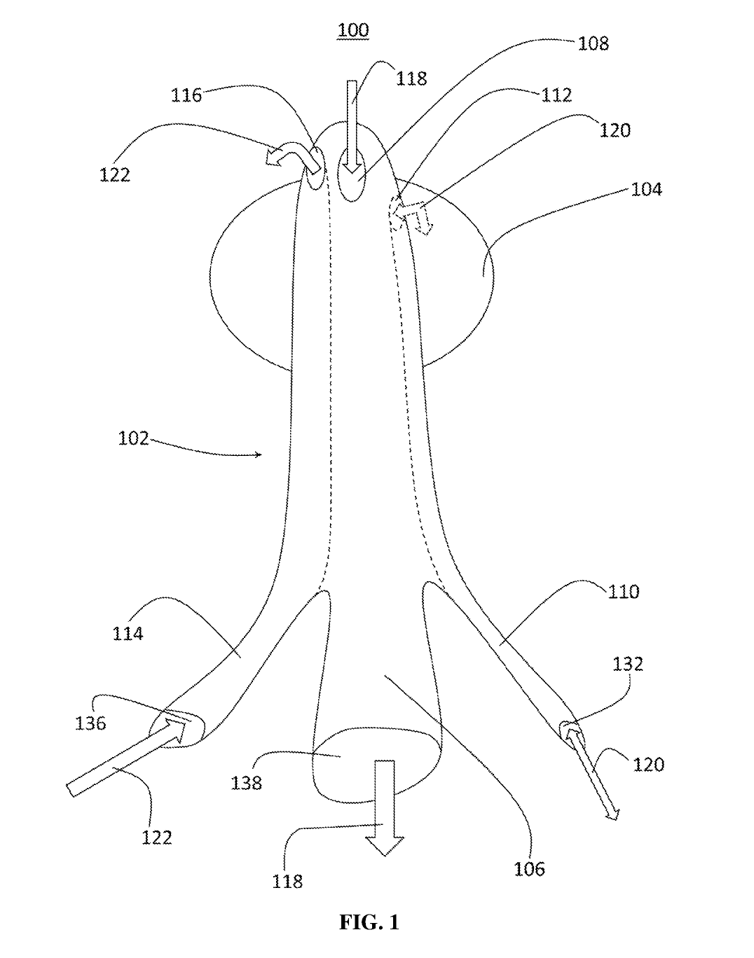

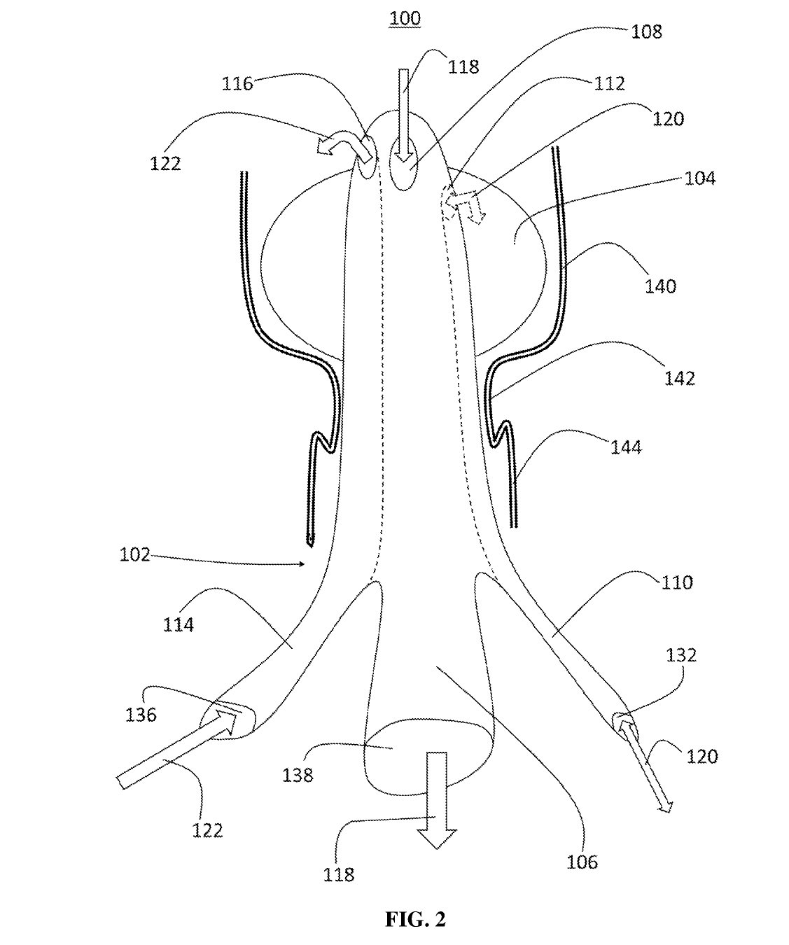

According to various aspects of the invention, the intracavitary devices of the present disclosure can comprise multiple configurations. For example, various exemplary embodiments of the inventive intracavitary devices are shown in FIGS. 1-2.

In further aspects, FIGS. 1-2 show various features and components of an exemplary intracavitary medical device in accordance with the present invention. The device comprises a tube 102 comprising a longitudinal body having opposed proximal and distal ends, and three lumens extending there between; and an expandable balloon region 104 located at the distal end of the tube 102. The three lumens comprise: a drainage lumen 106 having an opening 108 at a portion of the distal end of the tube and configured to allow fluid 118, such as blood and irrigation fluid, to drain from the body cavity into the drainage lumen 106; an inflation lumen 110 having an opening 112 connected to the interior of the balloon region 104 and configured to enable passage of a distending fluid 120 to control expansion and contraction of the balloon region 104; and an infusion-instillation lumen 114 having an opening 116 at a portion of the distal end of the tube 102 and configured to allow a fluid 122, such as a lavage or pharmaceutical compositions, to be introduced from the infusion-instillation lumen into the body cavity.

As shown in FIG. 2, the tube 102 travels from within the uterus 140, past the cervix 142, and exits the vagina 144. As it exits the vagina 144, the tube 102 branches externally/proximally in 3 ways, each branch corresponding to a lumen, and providing a proximal opening outside of the body for each lumen. In some aspect, the tube-catheter can have curved shape to assist in preventing device expulsion or sliding out.

The distending fluid 120 is infused through the proximal inflation lumen opening 132 into the interior of the balloon region 104 and expand the balloon. To this end, the expanded balloon exerts pressure upon the uterine cavity to stop bleeding by tamponade pressure upon the blood vessels, capillaries and tissue. At the same time, the expanding balloon can force collecting fluid 118, such as blood, from inside the uterine cavity into the distal drainage lumen opening 108, through the drainage lumen 106, and out through the proximal drainage lumen opening 138.

The infusion-instillation lumen 114 allows for infusion of an instillation fluid 122, such a medicated solution directly into the uterine cavity by injecting the medicine through proximal infusion-instillation lumen opening 136. The instillation fluid 122 passes through the infusion-instillation lumen 114 in one direction only. Instillations of drugs or irrigation, lavage, fluids into the proximal infusion-instillation lumen opening 136 empties directly into the uterine cavity without any connection with the balloon region 104.

In the present embodiment, the drainage lumen 106 and infusion-instillation lumen 114 are one-way passages. In various aspects, a one-way passage facilitates elimination of the collection fluids and can prevent infection inside the uterine cavity as indicated by the clinical condition. After completing the treatment and achieving the clinical goal, the distending fluid 120 is aspirated out and the tube 102 is removed.

The disclosed devices and methods have various advantages over existing devices and methods for treating body cavity or uterine disorders. In one aspect, the invention takes into consideration all the clinical conditions. Different clinical conditions require different sizes, which was taken into consideration in the present disclosure.

In another aspect, the present invention has a special passage for local topical injection which is not believed to be present in other devices. In further aspects, the addition of the special passage for irrigation/lavage can be different than the drainage passage. In still further aspects, this feature can be helpful in preventing any introduction of bacteria or infection in the uterine cavity. In yet further aspects, the additional passage can achieve the goal of irrigation lavage to prevent infection and can be used as a port to inject medications, such as antibiotics or uterotonic drugs to help contraction of the uterus. Topical medications can add to the treatment speed in the management of emergency cases.

In another aspect, the curved shape of the tube-catheter can assist preventing expulsion sliding out of the tube-catheter, which happens in at least 10% of clinical cases. Expulsion of the balloon can lead to failure of tamponade and continuing of the bleeding.

The disclosed devices and methods include at least the following aspects:

Aspect 1: An intracavitary medical device for insertion into a body cavity, the device comprising: a tube comprising a longitudinal body having opposed proximal and distal ends, and at least three lumens extending there between; and an expandable balloon region located at the distal end of the tube; wherein at least one lumen comprises at least one of: a drainage lumen which opens at a portion of the distal end of the tube and configured to allow fluid communication from a body cavity into the drainage lumen, an inflation lumen connected to an interior of the balloon region and configured to enable passage of a distending fluid to control expansion and contraction of the balloon region, and an infusion-instillation lumen which opens at a portion of the distal end of the tube and configured to allow a fluid to be introduced from the infusion-instillation lumen into the body cavity.

Aspect 2: An intracavitary medical device for insertion into a body cavity, the device comprising: a tube comprising a longitudinal body having opposed proximal and distal ends, and three lumens extending there between; and an expandable balloon region located at the distal end of the tube; wherein the three lumens comprise: a drainage lumen which opens at a portion of the distal end of the tube and configured to allow fluid communication from a body cavity into the drainage lumen; an inflation lumen connected to an interior of the balloon region and configured to enable passage of a distending fluid to control expansion and contraction of the balloon region; and an infusion lumen which opens at a portion of the distal end of the tube and configured to allow a fluid to be introduced from the infusion lumen into the body cavity.

Aspect 3: The device of any preceding aspect, wherein the device is configured for insertion into a body cavity.

Aspect 4: The device of any preceding aspect, wherein the device is configured to maintain a shape that conforms with the anatomical shape of the body cavity.

Aspect 5: The device of any preceding aspect, wherein the body cavity is a uterus or vagina.

Aspect 6: The device of any preceding aspect, wherein at least a portion of the tube body comprises a curved shape, a crescent shape, or an S shape, or a combination thereof.

Aspect 7: The device of any preceding aspect, wherein the device comprises a shape that conforms with the anatomical shape of a birth canal of a mammal.

Aspect 8: The device of any preceding aspect, wherein at least a portion of the tube body is comprised of a firm or rigid material.

Aspect 9: The device of any preceding aspect, wherein the device is comprised of latex, silicone, rubber latex, silicone coated latex, polyvinyl chloride, or polyurethane, or combinations thereof.

Aspect 10: The device of any preceding aspect, wherein the tube body has a length in the range of from about 20 cm to about 100 cm.

Aspect 11: The device of any preceding aspect, wherein the tube body has a diameter in the range of from about 24 French gauge to about 100 French gauge.

Aspect 12: The device of any preceding aspect, wherein the tube body diameter can be adjusted to accommodate an individual patient specific clinical condition.

Aspect 13: The device of any preceding aspect, wherein each lumen comprises at least one port or opening located at one end.

Aspect 14: The device of any preceding aspect, wherein at least one lumen comprises at least one port or opening located at both the proximal end and distal end.

Aspect 15: The device of any preceding aspect, wherein each lumen comprises a separate proximal opening.

Aspect 16: The device of any preceding aspect, wherein the diameter of the port is from about 0.1 mm to about 10 mm.

Aspect 17: The device of any preceding aspect, wherein at least one lumen has a distal opening (inside the body cavity), and a proximal opening (outside the body cavity).

Aspect 18: The device of any preceding aspect, wherein device comprises a separate port for filling the balloon region with a distending fluid, and a separate port for irrigation-lavage and/or pharmaceutical medications, topical treatment, injection, or infusion.

Aspect 19: The device of any preceding aspect, wherein each port is independent of and separate from the other ports.

Aspect 20: The device of any preceding aspect, wherein the tube comprises a plurality of lumens.

Aspect 21: The device of any preceding aspect, wherein each lumen is independent of and separate from other lumens.

Aspect 22: The device of any preceding aspect, wherein the diameter of the at least one lumen is from about 0.1 mm to about 10 mm.

Aspect 23: The device of any preceding aspect, wherein the diameter of the drainage lumen is from about 1 mm to about 10 mm.

Aspect 24: The device of any preceding aspect, further comprising a drainage port, designed for one way out exit drain.

Aspect 25: The device of any preceding aspect, wherein the opening of the drainage lumen comprises a Murphy's eye opening.

Aspect 26: The device of any preceding aspect, wherein the opening of the drainage lumen serves as passage out for uterine cavity fluid and bleeding collection.

Aspect 27: The device of any preceding aspect, further comprising a separate port for topical pharmaceutical medications or injection/infusion treatments.

Aspect 28: The device of any preceding aspect, wherein the drainage lumen is one way directed towards the uterine cavity only.

Aspect 29: The device of any preceding aspect, further comprising an internal/proximal opening for the separate port which directs filling solution to fill the balloon. This port connects directly to inside the balloon only, but not to the uterine cavity.

Aspect 30: The device of any preceding aspect, further comprising an opening connected to a separate port for drainage to rid of irrigation-lavage fluids in addition to bleeding which exits out of the patient body one way direction only.

Aspect 31: The device of any preceding aspect, further comprising an opening, external/distal which connects to the infusion/injection port, directed towards the uterine cavity where it delivers the topical pharmaceutical medications treatment and/or lavage solutions, directly into the uterine cavity, through the separate internal/proximal opening designed for that purpose.

Aspect 32: The device of any preceding aspect, the balloon region is configured to provide pressure tamponade; wherein the balloon region, when filled, takes the anatomical shape, size and volume of the uterine cavity, which can be variable in individual patient cases.

Aspect 33: The device of any preceding aspect, wherein the balloon is configured to be inflated with the distending fluid to a maximum volume from about 50 ml to about 750 ml.

Aspect 34: The device of any preceding aspect, wherein the tube body distal end is round and blunt, which helps prevent perforation injury of the uterine wall during insertion.

Aspect 35: The device of any preceding aspect, wherein the tube body has a predetermined size and a predetermined diameter.

Aspect 36: The device of any preceding aspect, wherein the balloon region has a predetermined size and a predetermined maximum volume.

Aspect 37: The device of any preceding aspect, wherein the tube body has a predetermined size and a predetermined diameter; and wherein the balloon region has a predetermined size and a predetermined maximum volume.

Aspect 38: The device of any preceding aspect, where the predetermined size of the tube body is from about 12 French gauge to about 48 French gauge.

Aspect 39: The device of any preceding aspect, where the predetermined diameter of the tube body is from about 24 French gauge to about 28 French gauge.

Aspect 40: The device of any preceding aspect, where the predetermined size of the balloon region is from about 20 ml to about 750 ml.

Aspect 41: The device of any preceding aspect, where the predetermined maximum volume of the balloon region is from about 1 ml to about 50 ml.

Aspect 42: The device of any preceding aspect, where the predetermined maximum volume of the balloon region is from about 51 ml to about 150 ml.

Aspect 43: The device of any preceding aspect, where the predetermined maximum volume of the balloon region is from about 151 ml to about 400 ml.

Aspect 44: The device of any preceding aspect, where the predetermined maximum volume of the balloon region is from about 401 ml to about 750 ml.

Aspect 45: The device of any preceding aspect, wherein the device is configured to treat a uterine disorder.

Aspect 46: The device of any preceding aspect, wherein the device is indicated for use in treating a uterine disorder.

Aspect 47: The device of any preceding aspect, wherein the uterine disorder comprises uterine bleeding, postpartum hemorrhage, uterine atony, abortion-miscarriage, placenta previa, recurrent uterine inversion, gestational trophoblastic disease, a hyperproliferative uterine disorder, uterine cancer, hydatidiform mole pregnancy, molar pregnancy, gestational trophoblastic disease, uterotonics, chorioamnionitis, endomyometritis, acute uterine inversion, sepsis/infection of uterine cavity including Obstetric or Gynecologic causes of intrauterine infection/sepsis such as Puerperal Sepsis, Postpartum Sepsis/Infection, Unsafe abortion sepsis/infection, Septic Abortion, Septic Miscarriage, Peritonitis, or Pelvic Inflammatory Disease (PID) or combinations thereof.

Aspect 48: The device of any preceding aspect, wherein the pharmaceutical composition comprises an effective amount of at least one compound selected from: an agent known to treat an infection or a pharmaceutically acceptable salt thereof; and an agent known to modulate the immune system or pharmaceutically acceptable salt thereof, an agent known to modulate hemostasis or pharmaceutically acceptable salt thereof, an agent known to treat cancer, an agent known to modulate hormones, and uterotonics, tocolytics, antibiotics, antiseptics, tranexamic acid, prostaglandins, or combinations thereof.

Aspect 49: A method for treating a uterine disorder in a subject, the method comprising: inserting and/or positioning a medical device according to any of the preceding aspects within a body cavity; and performing at least one of the following steps: expanding the balloon region within the body cavity, draining a fluid from the body cavity into the drainage lumen, and instilling a fluid or medication through the instillation lumen into the body cavity, thereby treating the subject for the uterine disorder.

Aspect 50: The method of any preceding aspect, wherein inserting comprises inserting the proximal internal end of the tube; wherein the empty balloon is filled with fluid solution by injecting fluid through the external/distal opening corresponding to the filling port, which ends at the internal/distal opening of the balloon region.

Aspect 51: The method of any preceding aspect, wherein the filled balloon region is configured to tamponade the inner wall of the cavity to pressure control a bleeding source of blood vessels.

Aspect 52: The method of any preceding aspect, wherein the filled balloon region is configured to direct blood and/or fluid collecting within the body cavity to exit through the distal drainage lumen opening.

Aspect 53: The method of any preceding aspect, further comprising topical treatment of the body cavity by instilling a pharmaceutical medication and/or lavage solutions using a separate port connected to the instillation lumen, wherein the external/distal corresponding opening and the internal/proximal corresponding opening directly into the uterine cavity.

Aspect 54: The method of any preceding aspect, wherein the topical medication is delivered directly into the uterine cavity.

Aspect 55: The method of any preceding aspect, wherein the drainage lumen is configured for the one way flow exiting and draining out fluid collections including bleeding but not for any other function; wherein it is not to be used for irrigation-lavage or any 2-way port direction.

Aspect 56: The method of any preceding aspect, wherein the device is configured to create a pressure tamponade to control bleeding and at the same time forcing collected blood or fluid towards the distal drainage lumen opening of the tube.

Aspect 57: The method of any preceding aspect, wherein the device is configured to provide at least one of the following functions: pressure-tamponade homeostasis function, drainage, lavage-irrigation, topical treatment, intracavitary drug delivery, topical drug delivery, intrauterine drug delivery, intrauterine drug treatment, tissue or blood sample collection, and combinations thereof.

Aspect 58: The method of any preceding aspect, wherein the device is configured to provide pressure-tamponade homeostasis function, drainage, lavage-irrigation, topical treatment, intracavitary drug delivery, topical drug delivery, intrauterine drug delivery, intrauterine drug treatment, tissue or blood sample collection, and internal cavity compression treatment evaluation.

Aspect 59: The method of any preceding aspect, wherein pressure-tamponade homeostasis comprises controlling bleeding originating from the uterine cavity.

Aspect 60: The method of any preceding aspect, wherein drainage comprises removing fluid and/or blood collection from the body cavity.

Aspect 61: The method of any preceding aspect, further comprising monitoring bleeding status.

Aspect 62: The method of any preceding aspect, wherein lavage-irrigation comprises instilling a fluid into the body cavity to facilitate clearing of collected fluid and/or blood in the body cavity.

Aspect 63: The method of any preceding aspect, further comprising clearing blood clots from the body cavity.

Aspect 64: The method of any preceding aspect, further comprising preventing and/or treating infections inside the uterine cavity.

Aspect 65: The method of any preceding aspect, wherein topical treatment comprises injection, infusion, and/or instillation of a pharmaceutical composition and/or medicated solution into the uterine cavity, as indicated by the individual patient clinical condition.

Aspect 66: The method of any preceding aspect, further comprising selecting a predetermined size and/or diameter of tube body based on the diagnosis.

Aspect 67: The method of any preceding aspect, further comprising selecting a predetermined size and/or volume of balloon region based on the diagnosis.

Aspect 68: The method of any preceding aspect, further comprising selecting a predetermined size and/or diameter of tube body based on the diagnosis; and selecting a predetermined size and/or volume of balloon region based on the diagnosis.

Aspect 69: A kit comprising: (a) a medical device according to any preceding aspect; and (b) instructions for using the medical device in connection with a uterine disorder.

Aspect 70: The kit of any previous aspect, wherein instructions for use comprise any step in a disclosed method.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An intracavitary medical device for insertion into a body cavity, the device comprising:
a tube comprising a longitudinal body having opposed proximal and distal ends, and at least three lumens extending there between; and
an expandable balloon region located at the distal end of the tube;
wherein at least one lumen comprises at least one of: a drainage lumen which opens at a portion of the distal end of the tube and configured to allow fluid communication from a body cavity into the drainage lumen, an inflation lumen connected to an interior of the balloon region and configured to enable passage of a distending fluid to control expansion and contraction of the balloon region, and an infusion-instillation lumen which opens at a portion of the distal end of the tube and configured to allow a fluid to be introduced from the infusion-instillation lumen into the body cavity.

2. The device of claim 1, wherein the device is configured to maintain a shape that conforms with the anatomical shape of the body cavity.

3. The device of claim 1, wherein the body cavity is a uterus or vagina.

4. The device of claim 2, wherein at least a portion of the tube body comprises a curved shape, a crescent shape, or an S shape, or a combination thereof.

5. The device of claim 4, wherein the device comprises a shape that conforms with the anatomical shape of a birth canal of a mammal.

6. The device of claim 5, wherein at least a portion of the tube body is comprised of a firm or rigid material.

7. The device of claim 1, wherein the tube comprises a plurality of lumens.

8. The device of claim 7, wherein the device comprises a separate lumen for filling the balloon region with a distending fluid, a separate lumen for infusion of irrigation lavage or pharmaceutical composition, and a separate lumen for drainage of fluids from the body cavity.

9. The device of claim 8, wherein the device is indicated for use in treating a uterine disorder.

10. The device of claim 9, wherein the uterine disorder comprises uterine bleeding, postpartum hemorrhage, uterine atony, abortion-miscarriage, placenta previa, recurrent uterine inversion, gestational trophoblastic disease, a hyperproliferative uterine disorder, uterine cancer, hydatidiform mole pregnancy, molar pregnancy, gestational trophoblastic disease, uterotonics, chorioamnionitis, endomyometritis, acute uterine inversion, uterine sepsis, infection of uterine cavity, Puerperal Sepsis, Postpartum Sepsis, Unsafe abortion sepsis, Septic Abortion, Septic Miscarriage, Peritonitis, or Pelvic Inflammatory Disease (PID) or combinations thereof.

11. The device of claim 10, wherein the device is configured to provide pressure-tamponade homeostasis function, drainage, lavage-irrigation, topical treatment, intracavitary drug delivery, topical drug delivery, intrauterine drug delivery, intrauterine drug treatment, tissue or blood sample collection, and internal cavity compression treatment evaluation.

12. The device of claim 11, wherein the pharmaceutical composition comprises an effective amount of at least one compound selected from: an agent known to treat an infection or a pharmaceutically acceptable salt thereof; and an agent known to modulate the immune system or pharmaceutically acceptable salt thereof, an agent known to modulate hemostasis or pharmaceutically acceptable salt thereof, an agent known to treat cancer, an agent known to modulate hormones, uterotonics, tocolytics, antibiotics, antiseptics, tranexamic acid, prostaglandins, or combinations thereof.

13. An intracavitary medical device for insertion into a body cavity, the device comprising:
a tube comprising a longitudinal body having opposed proximal and distal ends, and three lumens extending there between; and
an expandable balloon region located at the distal end of the tube;
wherein the three lumens comprise: a drainage lumen which opens at a portion of the distal end of the tube and configured to allow fluid communication from a body cavity into the drainage lumen; an inflation lumen connected to an interior of the balloon region and configured to enable passage of a distending fluid to control expansion and contraction of the balloon region; and an infusion lumen which opens at a portion of the distal end of the tube and configured to allow a fluid to be introduced from the infusion lumen into the body cavity.

14. A method for treating a uterine disorder in a subject, the method comprising:
Positioning an intracavitary device within a body cavity, the device comprising: a tube comprising a longitudinal body having opposed proximal and distal ends, and at least three lumens extending there between; and an expandable balloon region located at the distal end of the tube;
wherein at least one lumen comprises at least one of: a drainage lumen which opens at a portion of the distal end of the tube and configured to allow fluid communication from a body cavity into the drainage lumen, an inflation lumen connected to an interior of the balloon region and configured to enable passage of a distending fluid to control expansion and contraction of the balloon region, and an infusion-instillation lumen which opens at a portion of the distal end of the tube and configured to allow a fluid to be introduced from the infusion-instillation lumen into the body cavity; and
performing at least one of the following steps: expanding the balloon region within the body cavity, draining a fluid from the body cavity into the drainage lumen, and instilling a fluid or medication through the instillation lumen into the body cavity, thereby treating the subject for the uterine disorder.

15. The method of claim 14, wherein the device is configured to provide at least one of the following functions: pressure-tamponade homeostasis function, drainage, lavage-irrigation, topical treatment, intracavitary drug delivery, topical drug delivery, intrauterine drug delivery, intrauterine drug treatment, tissue or blood sample collection, and combinations thereof.

16. The method of claim 15, further comprising instilling a pharmaceutical medication and/or lavage solutions directly into the uterine cavity.

17. The method of claim 16, further comprising creating a pressure tamponade against the inner wall of the cavity to pressure control a bleeding source of blood vessels.

18. The method of claim 17, further comprising removing fluid and/or blood collection from the body cavity.

19. The method of claim 18, further comprising selecting a predetermined size and/or diameter of tube body based on the diagnosis; and selecting a predetermined size and/or volume of balloon region based on the diagnosis

20. The method of claim 19, wherein the uterine disorder comprises uterine bleeding, postpartum hemorrhage, uterine atony, abortion-miscarriage, placenta previa, recurrent uterine inversion, gestational trophoblastic disease, a hyperproliferative uterine disorder, uterine cancer, hydatidiform mole pregnancy, molar pregnancy, gestational trophoblastic disease, uterotonics, chorioamnionitis, endomyometritis, acute uterine inversion, uterine sepsis, infection of uterine cavity, Puerperal Sepsis, Postpartum Sepsis, Unsafe abortion sepsis, Septic Abortion, Septic Miscarriage, Peritonitis, or Pelvic Inflammatory Disease (PID) or combinations thereof.
